(19) United States
(12) Patent Application Publication
(10) Pub. No.: US 2025/0318788 A1
(43) Pub. Date: Oct. 16, 2025

(54) SYSTEMS, METHODS, AND COMPUTER-ACESSIBLE MEDIUM FOR PROVIDING PREDICTORS OF LOW RISK FOR DELIRIUM DURING ANESTHESIA EMERGENCE

(71) Applicants: The Trustees of Columbia University in the City of New York, New York, NY (US); Technische Universitát München, München (DE)

(72) Inventors: Rieke LUTZ, München (DE); Claudia Müller, München (DE); Srdjan Dragovic, München (DE); Frederick Schneider, München (DE); Katharina Ribbe, München (DE); Malte Anders, München (DE); Sebastian Schmid, München (DE)

(21) Appl. No.: 19/051,946

(22) Filed: Feb. 12, 2025

Related U.S. Application Data
(63) Continuation of application No. PCT/US2023/030062, filed on Aug. 11, 2023.
(60) Provisional application No. 63/397,667, filed on Aug. 12, 2022.
(60) Provisional application No. 63/459,294, filed on Apr. 14, 2023.

(51) Int. Cl. A61B 5/00; A61B 5/369

(52) U.S. Cl. CPC A61B 5/7275; A61B 5/369; A61B 5/4088

(57) ABSTRACT

Exemplary system, method and computer-accessible medium can be provided to monitor electroencephalography (EEG) data from the patient during an emergence from a general anesthesia previously provided to the patient, and predicting neurocognitive impairment based on a slope of EEG power. The neurocognitive impairment predicted can be delirium or it may be a predictor of long-term impariment such as Alzheimers. Further, the system and method can be used to direct a medical intervention based on the predicted neurocognitive impairment.

### CROSS REFERENCE TO RELATED APPLICATION(S)

This application relates to and claims the benefit of priority from U.S. Provisional Patent Application No. 63/397,667. filed on August 12. 2022, and U.S. Provisional Patent Application No. 63/459,294. filed on April 14. 2023. the entire disclosures of which are incorporated herein by reference in their entireties.

### FIELD OF THE DISCLOSURE

The present disclosure relates to predicting cognitive disorder, and more particularly to systems, method, and computer-accessible medium for predicting postoperative neurocognitive disorder during anesthesia emergence.

### BACKGROUND INFORMATION

With an aging population and an increasing number of diagnostic and operative interventions the occurrence of post-operative neurocognitive disorders is also growing. Various strategies have been suggested to help reduce the risk for perioperative neurocognitive disorders and to identify patients at risk; one of them is the use of intraoperative electroencephalography (EEG)-based monitoring. (See, e.g., Ref. 1). Processed EEG is widely used to monitor the level of anesthesia and has shown the potential to help physicians to identify patients at risk for perioperative neurocognitive disorders. (See, e.g., Ref. 1). Excessive anesthesia with burst suppression seems associated with a higher risk for a perioperative neurocognitive disorders especially when occurring in the maintenance period. (See, e.g., Refs. 2-4). Several different patterns of EEG trajectory have been defined for patients emerging from anesthesia. Like sleep patterns, the sequence of EEG patterns that the patient traverses during emergence seems to have a major impact on the perioperative cognitive state. (See, e.g., Refs. 5 and 6). While these trajectories were based on similar sleep states (‘delta dominant’ and ‘spindle dominant’) and help to describe the emergence EEG, they were arbitrarily defined. Embodiments herein focus on the question whether quantitative changes in EEG band power during the emergence phase can offer a simplified and low resource prognostic approach to identify patients at low risk for perioperative neurocognitive disorder according to their EEG, which could be easily applicable in a clinical setting of the OR and the PACU, in this case referred to specifically as a delirium according to previously published suggestions. (See, e.g., Ref. 7).

Thus, it may be beneficial to provide exemplary systems, methods and computer-accessible medium that can overcome at least some of the deficiencies described herein above.

### SUMMARY OF EXEMPLARY EMBODIMENT(S) AND METHODOLOGY

The following is intended to be a brief summary of the exemplary embodiments of the present disclosure, and is not intended to limit the scope of the exemplary embodiments.

To that end, it is possible to provide exemplary systems, methods and computer-accessible medium according to exemplary embodiments of the present disclosure, which can facilitate predicting postoperative mild or major neurocognitive disorder and delirium using patient EEG data.

The exemplary system, method, computer-accessible medium and apparatus according to the exemplary embodiments of the present disclosure can be used to monitor the level of anesthesia and has shown the potential to reduce the incidence for a perioperative neurocognitive disorder using EEG. While emergence trajectories that were identified post-hoc, show promising results in predicting a risk for a perioperative neurocognitive disorder, they are not easily transferable into an online predictive application. The exemplary system, method, computer-accessible medium and apparatus according to the exemplary embodiments of the present disclosure can provide a low-resource and easily applicable method to identify patients at high risk and low risk for a perioperative neurocognitive disorder, specifically delirium.

For example, 169 patients were included who underwent surgery with general anesthesia, maintained either with propofol, sevoflurane, or desflurane. The data were derived from a previously published study. Exemplary embodiments of the present disclosure can utilize, e.g., a single frontal channel and calculate the total and spectral band power from the EEG and calculate a linear regression model to observe the parameters' change during anesthesia emergence, described as slope. The slope of total power and single band power was correlated with the occurrence of a delirium.

Of 169 patients, 32 showed a delirium. Using the system, method, computer-accessible medium and apparatus according to the exemplary embodiments of the present disclosure, its was observed that patients whose total EEG power diminished the most during emergence were less likely to screen positive for delirium in the PACU. A significantly positive slope in total power and band power was associated with a higher risk (total: 2.83 [1.46 5.51]; alpha/beta band: 7.79 [2.24, 27.09]) for delirium. Furthermore, a significantly negative slope in multiple bands during emergence showed to be specific for patients without delirium and allowed to define a test for patients at low risk.

The system, method, computer-accessible medium and apparatus according to the exemplary embodiments of the present disclosure provide an easily applicable procedure to analyze a single frontal EEG channel and to identify patterns specific for patients at low risk for delirium. This approach may help to identify patients at risk and economize resources for patient screening.

In some exemplary aspects, the exemplary techniques according to the present disclosure described herein relate to a method for generating diagnostic data associated with a likelihood of developing neurocognitive impairment, comprising, e.g., monitoring electroencephalography (EEG) data from the patient during an emergence from a general anesthesia previously provided to the patient, and predicting neurocognitive impairment based on a slope of EEG power.

In some exemplary aspects, the exemplary techniques according to the present disclosure described herein relate to a system for generating diagnostic data associated with a likelihood of developing neurocognitive impairment, with the system comprising, e.g., a processor configured to (a) monitor electroencephalography (EEG) data from the patient during an emergence from a general anesthesia previously provided to the patient, and (b) predict neurocognitive impairment based on a slope of EEG power.

According to further exemplary embodiments of the present disclosure, a computer-readable non-transitory medium can be provided which can include computer-executable instructions that, when executed by at least one processor, perform procedures comprising, e.g., monitoring electroencephalography (EEG) data from the patient during an emergence from a general anesthesia previously provided to the patient, and predicting neurocognitive impairment based on a slope of EEG power.

In some exemplary aspects, the neurocognitive impairment can be delirium., a long-term impairment And/or, Alzheimer's. According to further exemplary embodiments of the present disclosure, the direction of a medical intervention can be based on the generated diagnostic data. Further, the medical intervention can be, e.g., an order for continued monitoring for neurocognitive impairment, and/or an order for a brain scan.

These and other objects, features and advantages of the exemplary embodiments of the present disclosure will become apparent upon reading the following detailed description of the exemplary embodiments of the present disclosure, when taken in conjunction with the accompanying claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A-1D are exemplary graphs and visualizations of an EEG analytical approach according to certain exemplary embodiments of the present disclosure;

FIGS. 2A-2E are exemplary graphs providing slope analysis for band power according to certain exemplary embodiments of the present disclosure;

FIGS. 3A-3E are exemplary graphs providing a starting power analysis for total and band power according to certain exemplary embodiments of the present disclosure;

FIGS. 4A and 4B are scatter plots for slopes in the alpha and beta bands according to certain exemplary embodiments of the present disclosure;

FIGS. 5A-5H are exemplary median spectrograms for absolute power approach according to exemplary embodiments of the present disclosure;

FIG. 6 is an illustration of an exemplary block diagram of an exemplary system in accordance with certain exemplary embodiments of the present disclosure;

FIG. 7 is an flow chart illustrating a patient selection and inclusion process of an exemplary system in accordance with certain exemplary embodiments of the present disclosure;

FIG. 8 is an illustration of an exemplary electrode layout on a patient in accordance with certain exemplary embodiments of the present disclosure;

FIGS. 9A-9D are exemplary graphs and visualizations of an EEG analytical approach according to certain exemplary embodiments of the present disclosure;

FIG. 10 is a graph of AUC curves by band power and age according to certain exemplary embodiments of the present disclosure;

FIG. 11 is a table providing exemplary results of an autocorrelation in the residuals with the Durbin-Watson test in accordance with certain exemplary embodiments of the present disclosure;

FIGS. 12A-12E are exemplary graphs of R-squared values for linear modeling comparing delirium to no delirium in total power and band power according to certain exemplary embodiments of the present disclosure;

FIGS. 13A-13E are exemplary graphs of slope analysis comparing delirium to no delirium in total power and band power according to certain exemplary embodiments of the present disclosure;

FIG. 14 is a scatter plot for slope in the alpha and beta bands according to certain exemplary embodiments of the present disclosure;

FIG. 15 is a table providing exemplary results of an exemplary test for patients at low risk for delirium in accordance with certain exemplary embodiments of the present disclosure;

FIG. 16 is an exemplary illustration of a generalized logistic regression model according to certain exemplary embodiments of the present disclosure;

FIG. 17 is a table illustrating exemplary results of a univariable analysis according to certain exemplary embodiments of the present disclosure;

FIG. 18 is a table illustrating exemplary group sizes and exemplary risk ratios for the slopes of total power and power for each frequency band according to certain exemplary embodiments of the present disclosure; and

FIG. 19 is a table illustrating exemplary group sizes and exemplary risk ratios for band power slope combinations for alpha (A) and beta (B) bands according to certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXAMPLE EMBODIMENTS

The following description of exemplary embodiments provides non-limiting representative examples referencing numerals to particularly describe features and teachings of different exemplary aspects and exemplary embodiments of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of embodiments, would be understood to be consistent with an application of the exemplary embodiments of the present disclosure.

### Exemplary Material And Methods

Exemplary design

The exemplary system, method, computer-accessible medium and apparatus according to the exemplary embodiments of the present disclosure can derive results from retrospective post-hoc analyses of a previously published dataset from patients with the goal to identify EEG signatures that correlate with delirium. (See, e.g., Ref. 8).

### Exemplary Patient Inclusion and Anesthesia Protocol

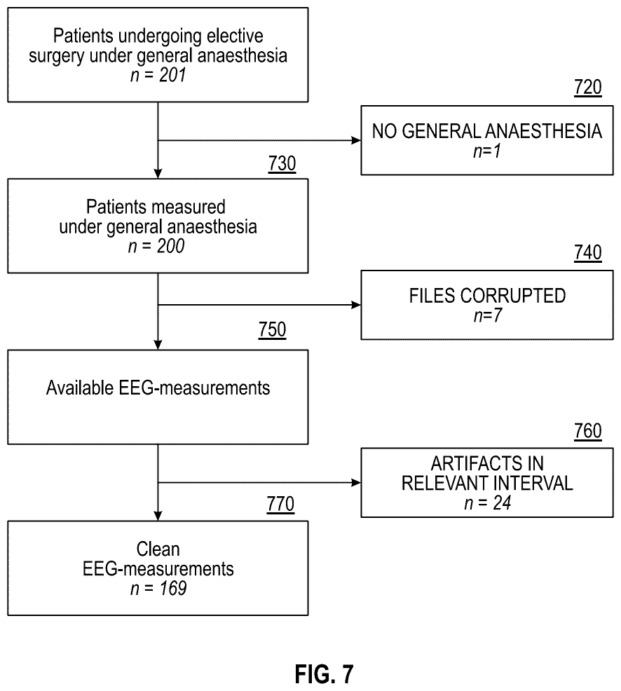

The exemplary system, method, computer-accessible medium and apparatus according to the exemplary embodiments of the present disclosure can include patients who underwent elective surgery in general anesthesia were older than 18 years of age and gave written and informed consent to participate in the study. For example, patients who underwent surgery in the 30 days prior, emergency interventions, suffered from psychiatric disorders or substance abuse were excluded from the study. Exemplary embodiments of the present disclosure performed exemplary procedures which can screen most or all patients preoperatively for delirium via Confusion Assessment Method for Intensive Care Units (CAM-ICU), a verified multistep procedure to identify patients with delirium. (See, e.g., Ref. 9). It is easily and quickly applied, has high inter-rater reliability and shows high sensitivity and specificity. (See, e.g., Ref. 10). Exemplary embodiments of the present disclosure can perform exemplary procedures or include exemplary systems or methods which can include 201 patients in total, although it should be understood that more or less patients may be included. Exemplary embodiments of the present disclosure can perform exemplary procedures or include exemplary systems or methods which can maintain anesthesia either with inhalational sevoflurane or desflurane, or intravenous propofol via syringe pump according to clinical standards. Exemplary embodiments of the present disclosure can perform exemplary procedures or include exemplary systems or methods which can achieve paralysis, if required, was achieved either with rocuronium or (in one case) with mivacurium. Sufentanil or remifentanil were used for intraoperative pain management. Exemplary embodiments of the present disclosure can perform exemplary procedures or include exemplary systems or methods which can be used to select dosage in accordance with clinical standards. Patient monitoring, e.g., according to exemplary embodiments of the present disclosure can be conducted according to the guidelines of the German society of anesthesiology (DGAI). FIG. 7 shows a flow diagram of a procedure or a method according to the exemplary embodiments of the present disclosure which can provide an exemplary resulting protocol of patient inclusion, where the boxes on the right represent the excluded patients from the original dataset. For example, as illustrated in Figure, in step 710, patients undergoing elective surgery under general anesthesia may be selected for inclusion. At step 720, patients who did not receive general anesthesia may be excluded. At step 730, relevant measurement may be made of the patients under general anesthesia. At 740, any corrupted data files may be removed from the process. At 750, EEG measurements may be made available resulting from the measurements of 730. If artifacts are found in the measurements for any patient during the relevant measurement/time interval, those may be excluded at 760. At 770, clean EEG measurements may be aggregated.

### Exemplary EEG Recording

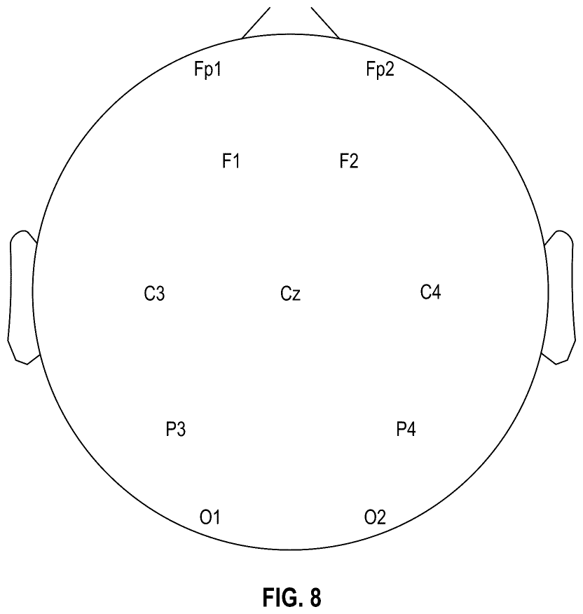

The exemplary system, method, computer-accessible medium and apparatus according to the exemplary embodiments of the present disclosure can rely on trained personnel to set up a 10 channel EEG recording prior to anesthesia induction, using non-invasive EEG electrodes applied according to the exemplary 10/20 system. For example, an electrode layout according to an exemplary embodiment of the present disclosure is shown in FIG. 8. A reference electrode Cz can be provided. The exemplary system, method, computer-accessible medium and apparatus according to the exemplary embodiments of the present disclosure can confirm correct positioning of electrodes relative to the reference electrode each time a patient is moved, after induction, and before emergence. For example, FIG. 8 illustrates the relative positioning of electrodes Fp1, Fp2, F1, F2, C3, C4, P3, P4, O1, and O2. Signal quality is monitored in exemplary embodiments throughout the whole intervention. The EEG can be recorded with the NIM-Eclipse intraoperative neuromonitoring system (Medtronic, Dublin, Ireland), in some embodiments with a 250 Hz sample rate and a 1Hz hardware high pass filter. Data can be stored in the native .eeg format from Medtronic.

### Exemplary Emergence and Cognitive Assessment

According to the exemplary embodiments of the present disclosure, after terminating anesthetic delivery, patients can be verbally addressed at regular intervals until they respond purposefully. For example, addressing the patients can begin either when the end tidal alveolar gas concentration reaches the minimum alveolar concentration (MAC) awake (0.35% for sevoflurane, 0.55% for desflurane) or 5 minutes after terminating propofol delivery. Patients can be addressed at 1-minute intervals until they reach a score of greater than, e.g., 2 on the Observer's Assessment of Alertness/Sedation (OAA/S) scale. The OAA/S is a scale used to measure the level of alertness in sedated patients and consists of the following 4 categories: responsiveness, speech, facial expression, and eye contact. The scale ranges from, e.g., 1 (deep sleep) to 5 (alert), where a score of 3 represents a response with eyes opening only after name is called loudly. (See, e.g., Ref. 14). The exemplary system, method, computer-accessible medium and apparatus according to the exemplary embodiments of the present disclosure can be used to define this as the end of emergence. Embodiments may assess patients at, e.g., 15 and 60 minutes later in a recovery room to check for delirium using the CAM-ICU. The exemplary system, method, computer-accessible medium and apparatus according to the exemplary embodiments of the present disclosure can define patients as positive for delirium in the PACU, if they scored positive on the CAM-ICU at either (or both) 15 or 60 minutes.

### Exemplary EEG Pre-Processing

Exemplary Data import

The exemplary system, method, computer-accessible medium and apparatus according to the exemplary embodiments of the present disclosure can process EEG data with MATLAB 2020a (Natick, Massachusetts: The Math Works Inc.) and the MATLAB toolbox eeglab., (See, e.g., Ref. 12). The exemplary system, method, computer-accessible medium and apparatus according to the exemplary embodiments of the present disclosure can import data import into MATLAB using custom routines.

### Exemplary Data Clean-Up and Power Spectral Density

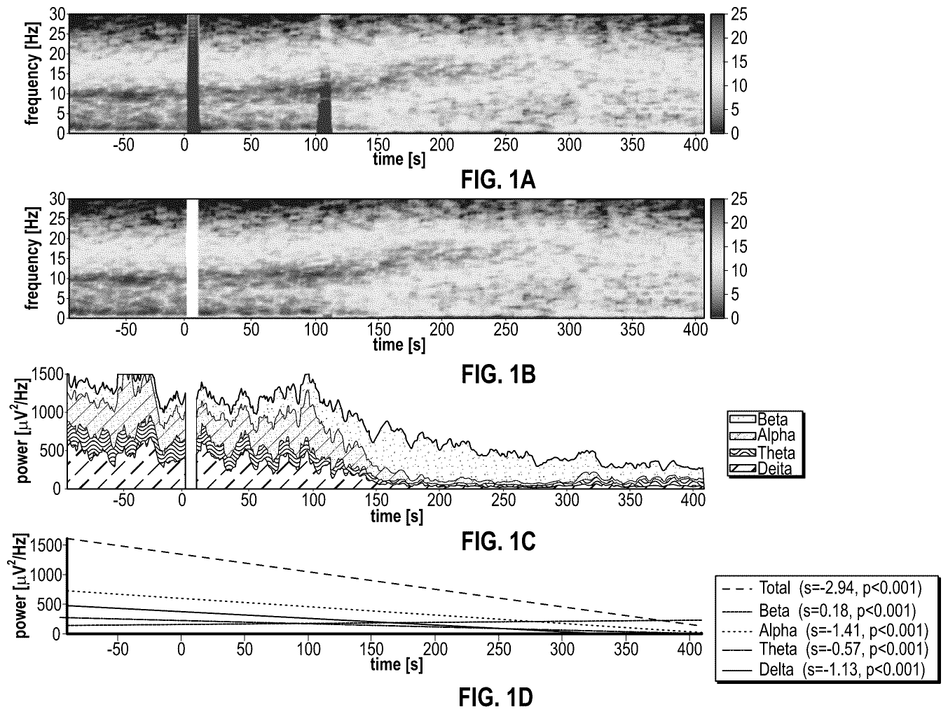

A single frontal channel (Fp2-Fz) can be chosen by exemplary embodiments for this analysis because it may reflect the current layout of most commercial EEG-based monitoring devices. Exemplary embodiments can first apply a low pass filter at 47 Hz using the eeglab function eegfilt. This may be done for two reasons. Firstly, to eliminate the 50 Hz line noise and secondly to remove high frequent signal distortions like the EMG that become dominant in higher frequencies but overlap with the EEG spectrum. (See, e.g., Refs. 13 and 14). The exemplary system, method, computer-accessible medium and apparatus according to the exemplary embodiments of the present disclosure can clean artifacts from the EEG in. e.g., two steps. In the first exemplary step/procedure, it is possible to utilize an automated artifact subspace reconstruction with clean_rawdata and set the artifact subspace reconstruction parameter to 25 standard deviations as suggested for automated protocols. (See, e.g., Ref. 15). The other options of the function can be turned off. For the emergence period density spectral arrays can be created using the pwelch function with NFFT=512 over 10 s EEG segments with a Is shift. In exemplary embodiments of the present disclosure, the density spectral array can be calculated or otherwise determined for the emergence phase with t0 starting at 90 s before start of emergence representing the maintenance phase and t1representing end of emergence (OAA/S>2). An examplary density spectral array derived from raw EEG data is shown in an illustration of FIG. 1A.

### Exemplary Z-Scoring for Further Artifact Removal

In an exemplary embodiment of the present disclosure, after visually inspecting the 193 density spectral arrays after artifact subspace reconstruction, e.g., 83 data sets had to undergo a second step of artefact rejection because of clearly identifiable artifacts. Visual inspection can be focused on excessive blue or red coloring (e.g., darker or brighter shades in FIG. 1A) in the density spectral array plots. Red coloring (e.g., dark shade) can indicate a very high power caused by artifacts and blue may represents very low power caused by zero lines. According to certain exemplary embodiments of the present disclosure, it is possible to calculate or otherwise determine Z-scores for total power of every column of the density spectral array, and those with a score, e.g., greater than 3 (e.g., greater than 2 in some cases) can be excluded from the array. The extra step/procedure of lowering the z-score to about less than 2 may be performed if the higher boundary does not exclude all remaining artifacts still visible in the resulting density array.

The exemplary system, method, computer-accessible medium and apparatus according to the exemplary embodiments of the present disclosure can provide a data exclusion not exceeding, e.g., 5%. An exemplary cleaned density spectral array is shown in FIG. 1B. The remaining 110 data sets of an exemplary embodiment may not undergo the second stage of artefact rejection. The cleaned density spectral arrays can then again be visually inspected and compared to the original density spectral array for errors in the routine and remaining artifacts. According to one exemplary embodiment, from the exemplary 83 data sets, a total of 24 data sets were excluded as they either were too contaminated by remaining artifacts or more than 10% of data was missing in the investigated interval because of technical issues. The exclusion can be performed by two different entities, which may be blinded to the delirium scores. The final data set for one exemplary embodiment consisted of 169 patients eligible for further analysis.

### Exemplary Calculating Trajectories

### Exemplary Band Power

From the cleaned density spectral arrays exemplary embodiments can calculate the EEG band power for the delta band (e.g., 1-4 Hz), the theta band (e.g., 4-8 Hz), the alpha band (e.g., 8-15 Hz), and the beta band (e.g., 15-47 Hz). The respective exemplary band power can be calculated by numerical integration using the chained trapezoidal rule (trapz function). According to the exemplary embodiments of the present disclosure, it is possible to define the total EEG power as the cumulative sum of the power in all frequency bands. The exemplary course of band power is exemplarily shown in an exemplary graph of FIG. 1C.

### Exemplary Linear Regression

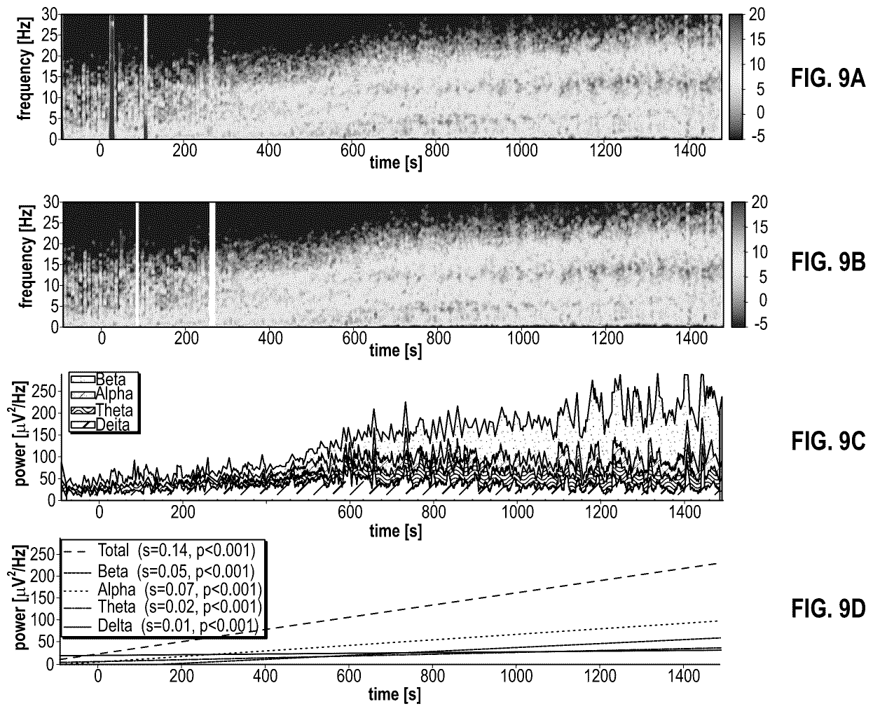

An exemplary linear regression of the change in total power and absolute band power with time during emergence can be calculated or otherwise determined by the system. method and computer-accessible medium according to the exemplary embodiments of the present disclosure using the fitlm function, using the total power and band power values for each second. The possible exemplary results can fall into 3 categories, e.g., either a significantly rising or falling power, represented by an either positive or negative slope and a p-value<0.05; or no significant change in power and a p-value>0.05 (see exemplary graph of FIG. 1D). An example for a patient with delirium is shown in exemplary graphs and illustration of FIGS. 9A-9D. In particular, FIG. 9A illustrates an exemplary density spectral array derived from the original recording, i.e., the raw; uncleaned EEG for the delirium positive patient. FIG. 9B shows an exemplary density spectral array after pre-processing including artifact subspace reconstruction and z-score based artifact rejection. FIG. 9C illustrates a graph of an exemplary course of the EEG band powers of the four main frequency bands. Finally. FIG. 9D shows a graph of exemplary band slopes derived from the linear regression for the different bands with the corresponding p-values.

In exemplary embodiments of the present disclosure, tests for autocorrelation can be evaluated with the Durbin-Watson test using the dwtest function. Quality of fit can be evaluated by reporting the R² values. For an exemplary test for no-delirium, the exemplary results from the linear regression can be discretized and patients can then be classified according to the sign of the slope in total power and the different bands as positive (+), negative (−) or not significant (n.sig) for the corresponding bands. System, method and computer-accessible medium according to the exemplary embodiments of the present disclosure can, e.g., only assess the signs of the value and disregard further consideration of absolute values.

### Exemplary Missing Data

The exemplary system, method, computer-accessible medium and apparatus according to the exemplary embodiments of the present disclosure can conduct a complete case analysis. Corrupted eeg datasets can excluded from a study. Severe artifacts during emergence can either be cleaned to a satisfactory level or be excluded by two independent and blinded investigators. Excluded datasets with artefacts can be included in a sensitivity analysis but may be excluded for further multiband analysis. Exemplary embodiments may have no missing epidemiological data.

### Exemplary Risk Groups

The exemplary system, method, computer-accessible medium and apparatus according to the exemplary embodiments of the present disclosure may classify patients according to the combinations of signs of the slope in the different bands, specifically alpha and beta. Three exemplary options can exist for alpha and beta: positive, negative and not significant. 9 possible classification options can result, e.g., A−/B− representing a negative slope in both the alpha and the beta band, A+/B+ representing a positive slope in both the alpha and beta band.

### Exemplary Statistical Analysis

For example all (statistical) analyses, according to the exemplary embodiments of the present disclosure, can be conducted with MATLAB 2019a. Group comparisons can be performed using the non-parametric two-sided Wilcoxon rank sum test, as normal distribution may not always be assumed. When comparing contingency data, Fisher's exact test can be used. Exemplary embodiments chose Fisher's test as certain groups were small. The exemplary results can be given either as the median and the first and third quartile or the number and percentage of patients with or without a positive CAM-ICU test. In addition, the calculated p-values, Fisher's exact test statistic and the reference group for the Fisher's test can be noted. System, method and computer-accessible medium according to the exemplary embodiments of the present disclosure can support the p-values by effect sizes, i.e., the risk-ratios with 95% confidence intervals or the area under the receiver operating curve (ROC). An exemplary AUC calculation with 10-k bootstrapped 95% confidence intervals can be conducted, according to exemplary embodiments, using the MATLAB-based MES toolbox. (See, e.g., Ref. 16). The statistical measures and predictive values for the preliminary test for no-Delirium, according to exemplary embodiments of the present disclosure, can also be 10-k bootstrapped for internal validation. The exemplary system, method and computer-accessible medium according to the exemplary embodiments of the present disclosure can conduct sensitivity analysis by including the data from all patients before exclusion by the two different independent investigators and comparing it to the dataset after exclusion.

### Exemplary Covariate Analysis

The exemplary system, method and computer-accessible medium according to the exemplary embodiments of the present disclosure can check for potential association between the variables of the univariate analysis and the categories of slopes from the linear regression using the non-parametric two-sided Wilcoxon rank sum test. Exemplary embodiments may use the ranksum function. In some embodiments, if the p-value is lower than 0.05, the Slope Classification and prediction can be corrected for the variable. If not, the variable can be included as an independent covariate in a generalized logistic regression model. The exemplary system, method and computer-accessible medium according to the exemplary embodiments of the present disclosure can calculate or otherwise determine a generalized logistic regression model using the fitglm function.

### Exemplary Results

In one exemplary embodiment of the present disclosure, out of 201 patients included in an original study, 169 were eligible for analysis and were included. In one exemplary embodiment 7 data files out of 201 were corrupted and could not imported, one patient did not receive general anesthesia, and 24 still contained artifacts in the relevant interval after the artifact removal process.

### Exemplary Demographics and Univariate Analysis

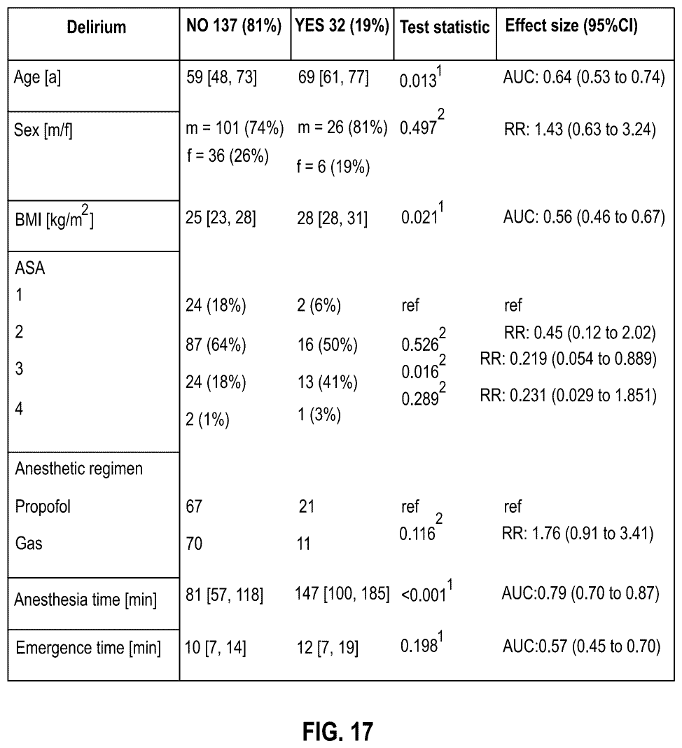

The table shown in FIG. 17 provides exemplary results, according to an exemplary embodiment of the present disclosure, of the univariable analysis for patients who developed a delirium (n=32 (19%)) and those who did not (n=137 (81%)). Table shown in FIG. 17 provides exemplary results as median, first and third quartile or number and percentage in the group. For example, test statistics according to exemplary results of FIG. 17 are either given as p-values calculated with the Wilcoxon-Rank-Sum test (1) or Fisher's exact test statistic (2) and effect sizes are given as the Area-Under-the-Curve or Risk-Ratio with the corresponding confidence interval. Further, in FIG. 17, italicized values can be statistically significant. The exemplary system, method and computer-accessible medium according to the exemplary embodiments of the present disclosure can reveal that patients with a delirium were significantly older (p=0.013). This is consistent with previously published results. (See, e.g., Ref. 17). Furthermore, the exemplary system, method and computer-accessible medium according to the exemplary embodiments of the present disclosure can establish that patients with delirium had a significantly higher BMI (p=0.021).

The exemplary system, method, computer-accessible medium and apparatus according to the exemplary embodiments of the present disclosure can provide a significant positive correlation between anesthesia time and deliriums, which is in line with previously published studies. (See, e.g., Ref. 6). ASA 3 can be associated with a statistically significant higher risk for Delirium. There may be no statistically significant association between sex, anesthetic regimen, and emergence time and delirium. According to one exemplary embodiment of the present disclosure, the group excluded from EEG analysis (n=31 (16%)) did not show a statistically significant different incidence of delirium (n=4 (13%)) as the included group (Fisher's exact test statistic=0.66, not significant at p>0.05). Also according to one exemplary embodiment of the present disclosure, there may be no demographic data missing for the included patients, and no follow-up after a stay in the PACU.

### Exemplary Linear Regression

### Exemplary Total Power and Single Band Power

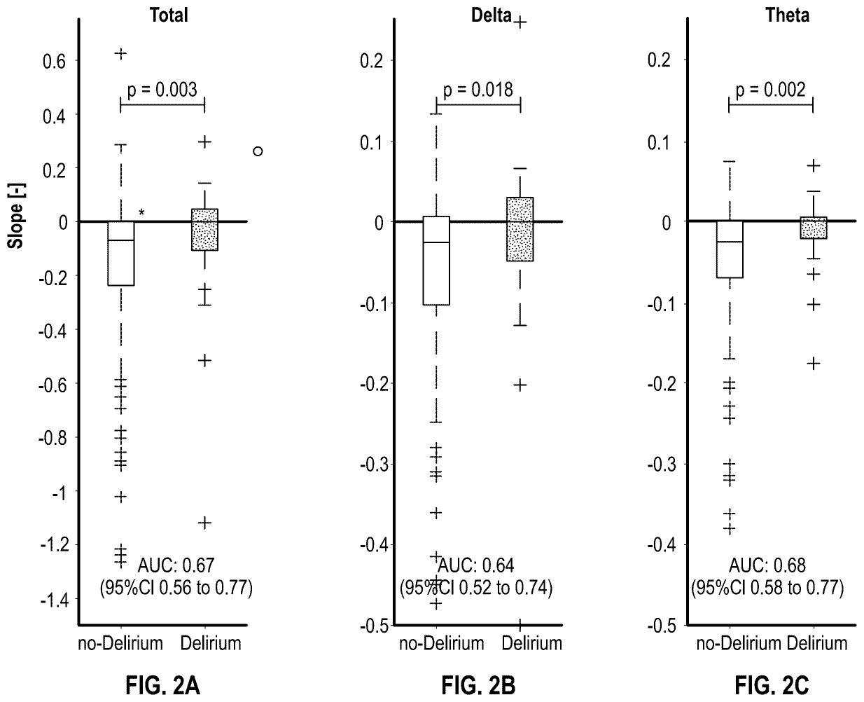

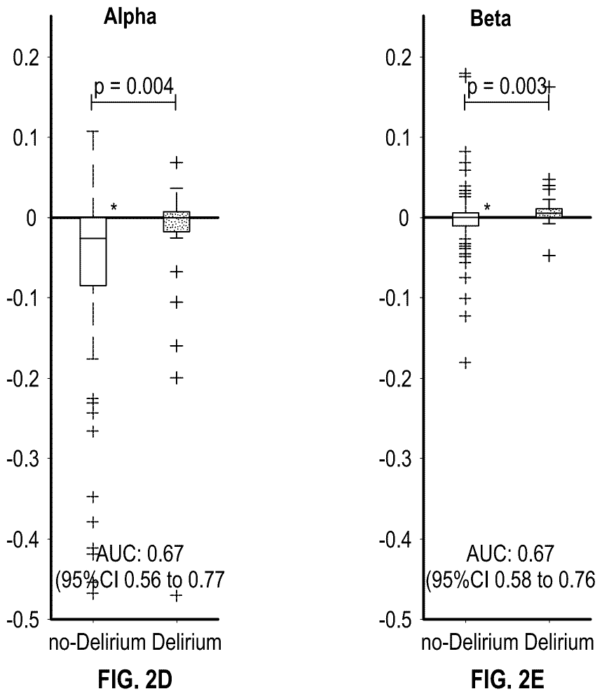

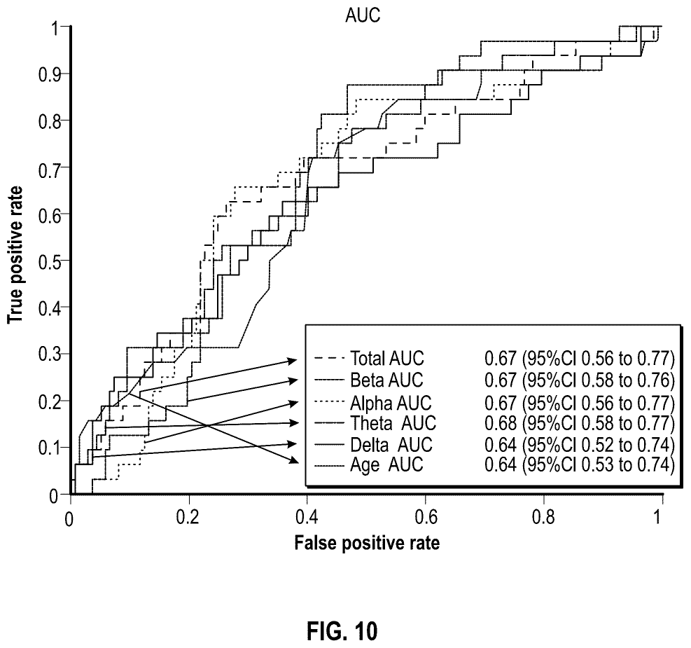

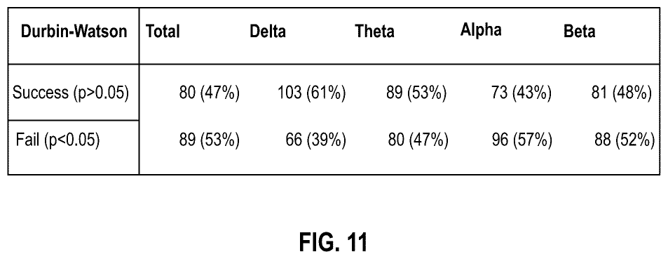

FIGS. 2A-2E show exemplary graphs or box-plots for the emergence slope distributions for total power (see, e.g., FIG. 2A) and the power in the different bands (see, e.g. FIGS. 2B-2E, according to the exemplary embodiments of the present disclosure. The p-values in these exemplary graphs of FIGS. 2A-2E can be calculated using the Wilcoxon-Rank-Sum Test. All groups in the exemplary graphs can be significantly different and can be supported by effect sizes in the form of an AUC and the corresponding 95% confidence interval. As evidenced in such exemplary graphs, slopes in the no-Delir group are more often negative than positive for total power and all band powers. The exemplary system, method and computer-accessible medium according to the exemplary embodiments of the present disclosure can reveal that patients who did not develop a delirium tended to show a steeper negative slope for total power and across all bands during the emergence phase than patients who developed a delirium. Furthermore, in total power and in all bands the slopes were more often negative than positive in the group without a delirium. Effect sizes in the form of AUCs can range between 0.64 (95% CI: 0.52 to 0.74) for the delta band and 0.67 (95% CI: 0.58 to 0.77) for the alpha band. All AUC curves including age are shown in FIG. 10. The exemplary results of the analysis for autocorrelation in the residuals with the Durbin-Watson test are shown in the table of FIG. 11

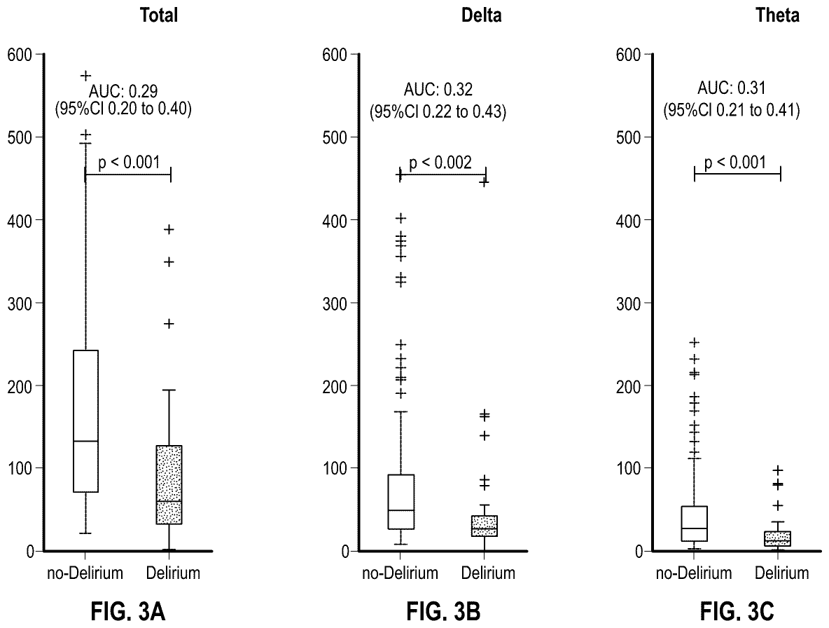

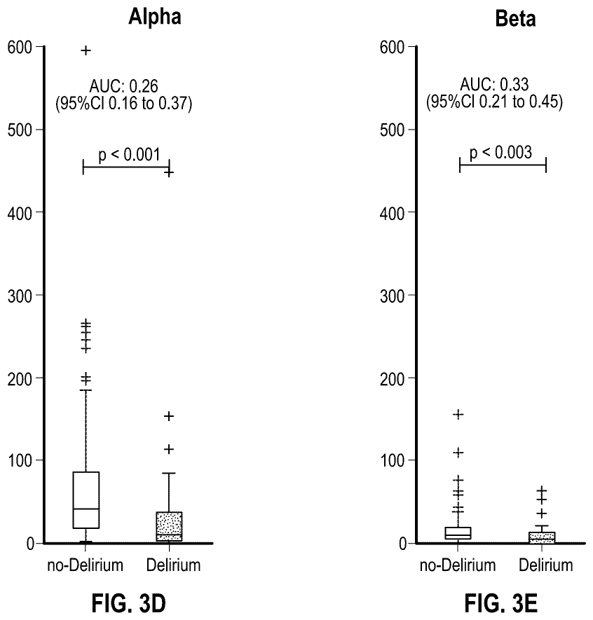

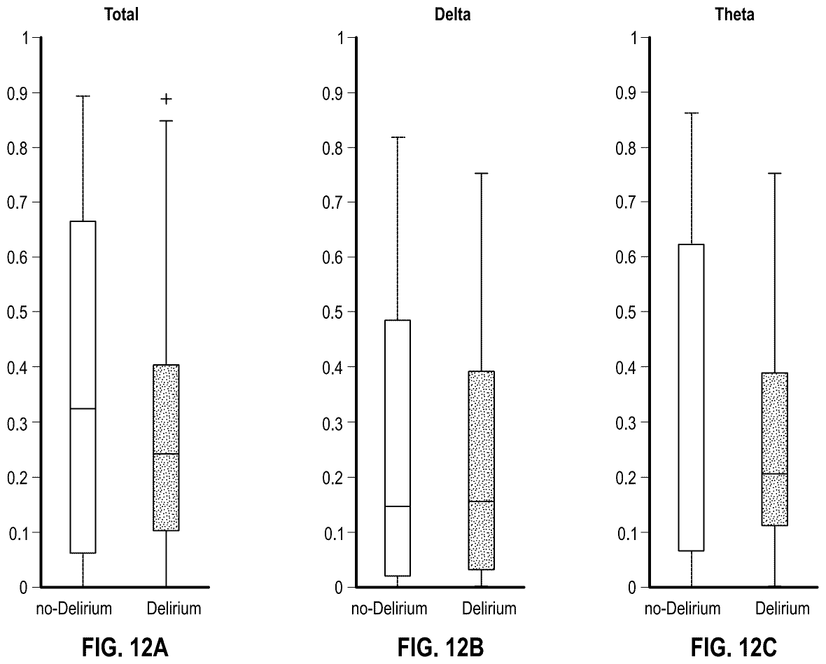

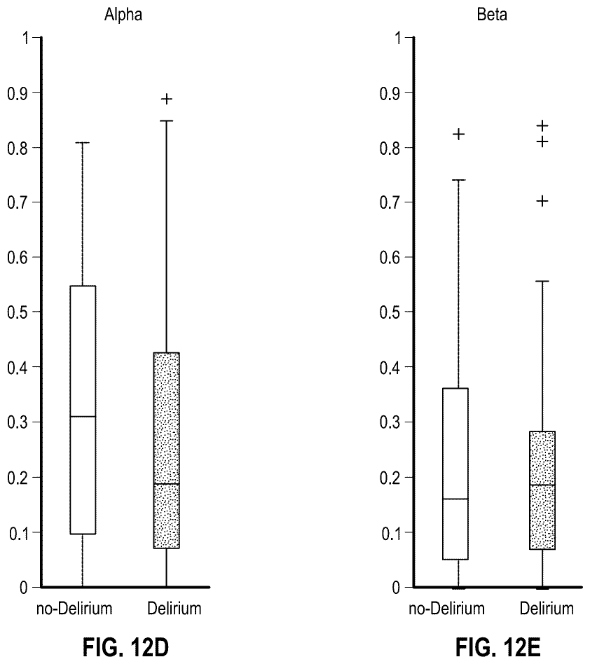

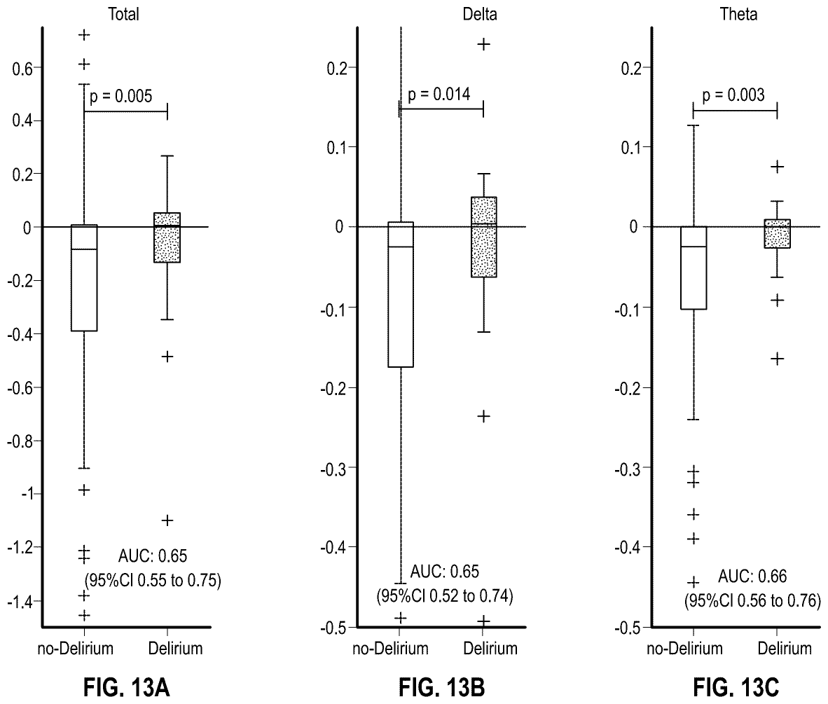

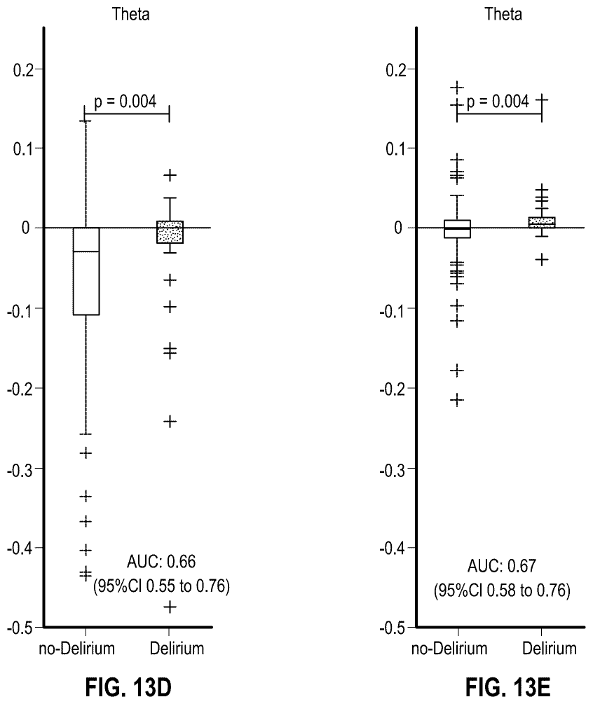

FIGS. 3A-3E show exemplary graphs or box-plots for the distributions for total starting power (see, e.g., FIG. 3A) and the starting power in the different bands (see, e.g., FIGS. 3B-3E, according to exemplary embodiments. For example, according to FIGS. 3A-3E. P-values for the exemplary graphs can be calculated using the Wilcoxon-Rank-Sum Test. All groups in the exemplary graphs can be significantly different, and can be supported by effect sizes in the form of an AUC and the corresponding 95% confidence interval. Similar to the exemplary slope values in FIG. 2A-2E, the system, method and computer-accessible medium according to the exemplary embodiments of the present disclosure can indicate that patients who did not develop a delirium may have a significantly higher starting power across all bands and in total power than those who did develop a delirium. This is in accordance with previously published results by Lutz et al. (See, e.g., Ref. 8). The corresponding R² values for linear modelling comparing no-Delirium with Delirium for each band power and total power are reflected in the exemplary graphs and/or box plots of FIGS. 12A-12E. For example, using an exemplary sensitivity analysis. FIGS. 13A-13E illustrate exemplary graphs and/or box-plots similar to those shown in FIGS. 3A-3E, with all datasets included (n=192). There may be no or little significant differences between the two groups.

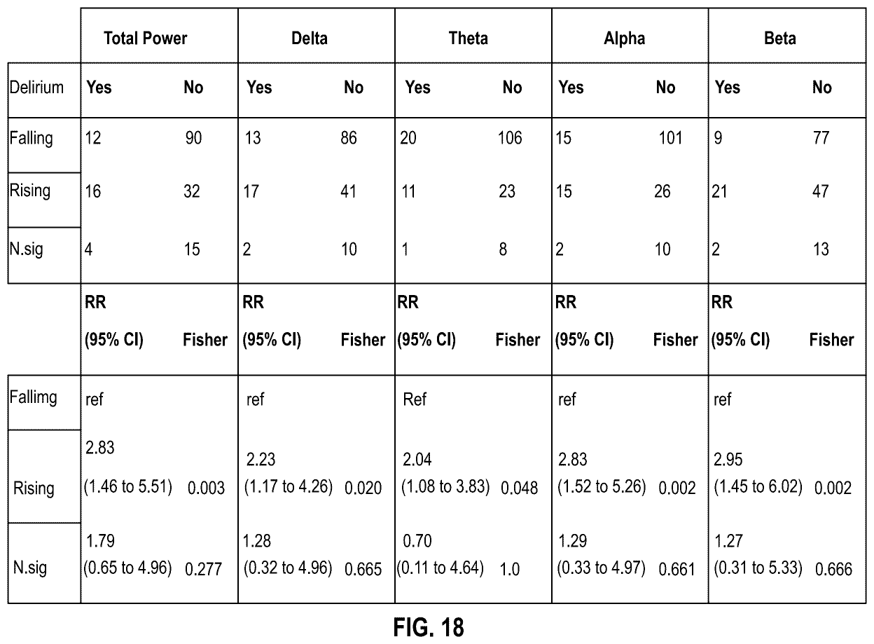

The table shown in FIG. 18 provides the exemplary group sizes and the risk ratio for delirium for patients with either significantly rising total power or band power and for patients without significant change in total power or band power during emergence, according to exemplary embodiments. The exemplary system, method and computer-accessible medium according to the exemplary embodiments of the present disclosure can show that patients with increasing power either across the EEG bands, or just in singular bands, can have an approximately two-fold risk to develop a delirium when compared to patients with decreasing EEG power. There may be no significant difference in risk between patients with decreasing EEG power and patients without significant change of EEG power during emergence.

### Exemplary Multi Band Analysis

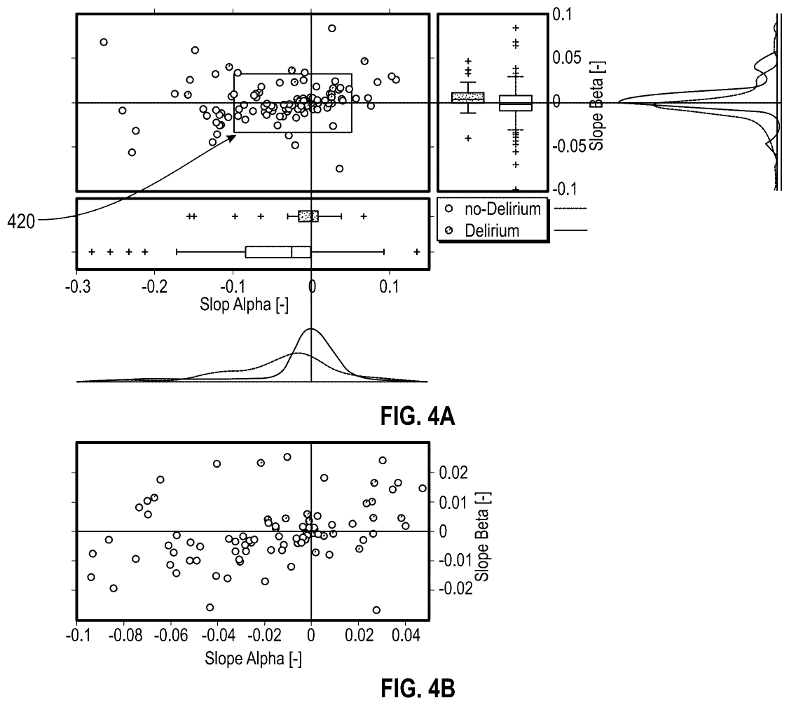

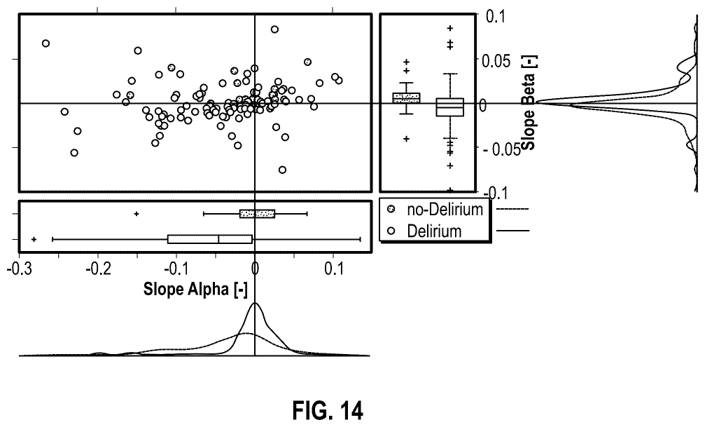

FIGS. 4A and 4B show exemplary scatter plots of slope value pairs for alpha band and beta band power for each patient depending on delirium status, according to the exemplary embodiments of the present disclosure. For example, in the lower left quadrant where alpha and beta power are decreasing, there are mostly non-delirious patients. This is also shown in the distribution plots of FIG. 4A, where the mean slope value for both alpha and beta slope is negative. A detailed exploded view of the dashed box 420 of FIG. 4A is shown in FIG. 4B. An exemplary supplemental scatter plot shown in FIG. 14 illustrates the distributions for alpha and beta for all patients, including those with no significant change in power in beta and alpha. Furthermore, FIGS. 2D and 2E show a negative median value for both bands.

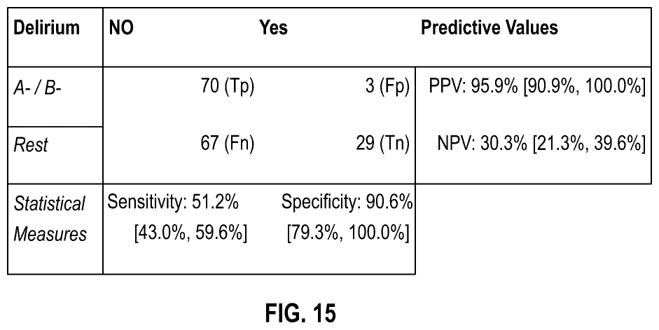

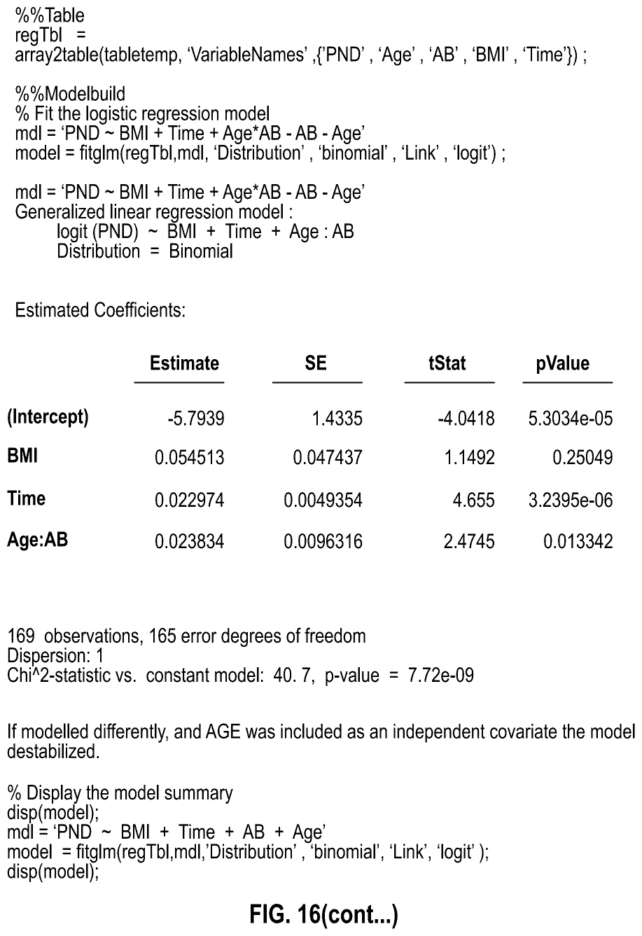

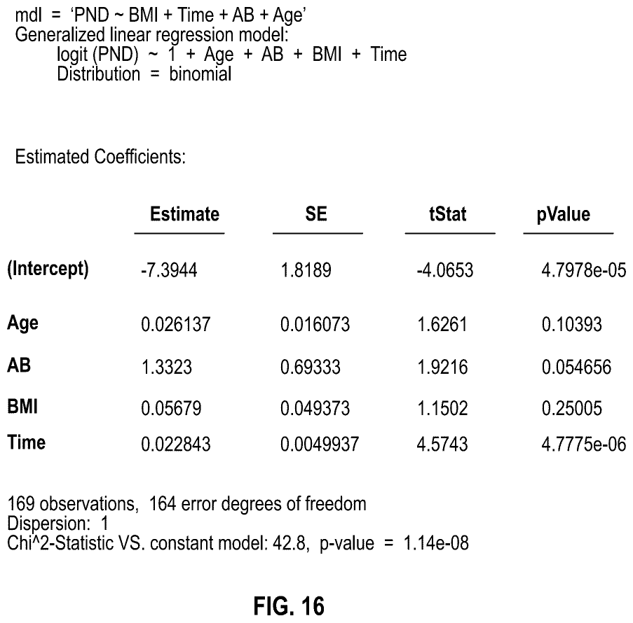

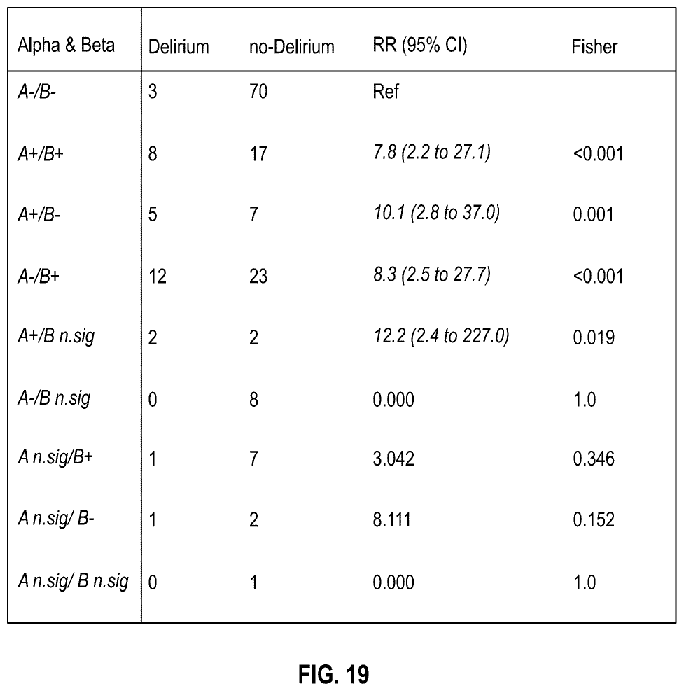

The table illustrated in FIG. 19 indicates the combinations of changes in EEG band power, the corresponding risk ratios, and Fisher's exact test statistics, according to the exemplary embodiments of the present disclosure. A falling band power in both the alpha and beta band can be associated with the lowest risk for delirium and was therefore set as reference. This can mean that a negative slope in both the alpha and beta band can be highly specific for patients who wake up without a delirium. In certain exemplary embodiments of the present disclosure, patients who showed an increase in band power in at least one band can have a significantly higher risk for delirium. A test for patients at low risk for delirium is shown in the table of FIG. 15. The exemplary calculated test shows a specificity of 90.6%, a sensitivity of 51.2%, a PPV of 95.9%, a NPV of 30.3%, and a p-value<0.001. The corresponding 10-k bootstrapped AUC of an exemplary embodiment equals 0.69 (95% CI: 0.64 to 0.74). A covariate analysis performed using the exemplary system, method and computer-accessible medium according to the exemplary embodiments of the present disclosure so as to test for confounding this test is shown in FIG. 16. In the preliminary testing for choosing the variables, the exemplary system, method and computer-accessible medium according to the exemplary embodiments of the present disclosure revealed no correlation between Time of Anesthesia and Slope A−/B− (p=0),834), BMI and Slope (p=0.834). In some embodiments, these variables can be included as independent covariates in the generalized logistic regression model. There can be significant correlation between Age and Slope (p=0.027). In a final model of an exemplary embodiment, the Slope can be included adjusted for Age, BMI and Time of Anesthesia can be included as independent covariates. The results of an exemplary embodiment, including the models, from an analysis are reflected in FIG. 16.

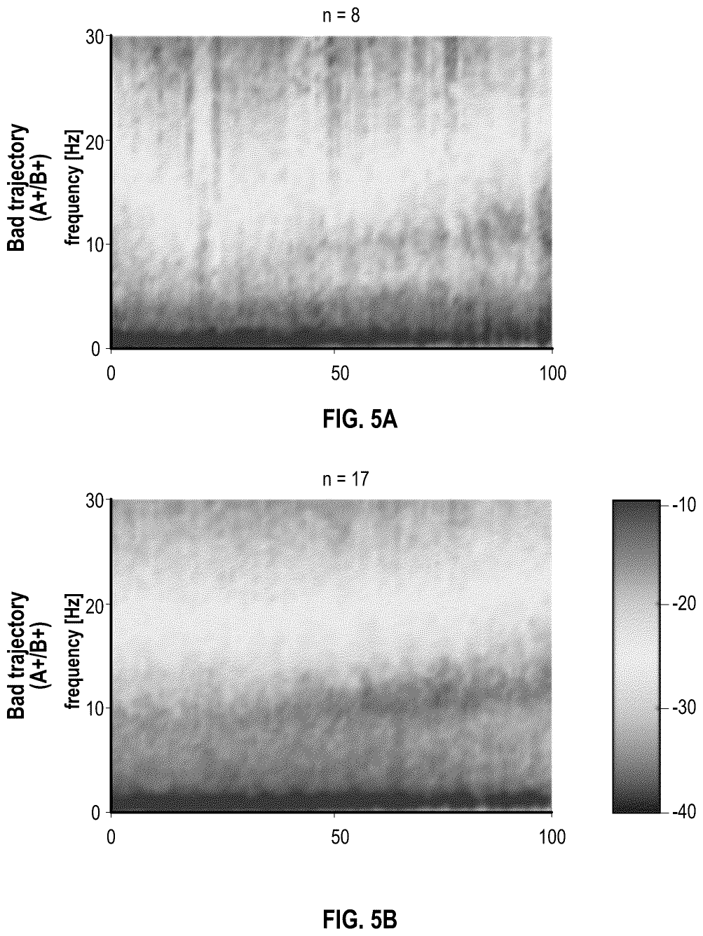

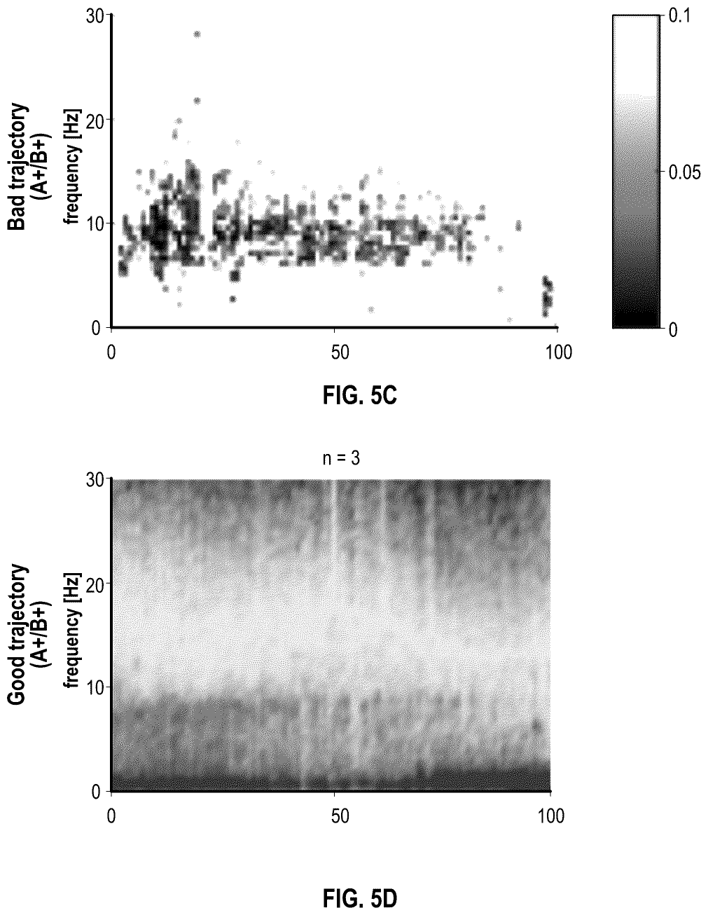

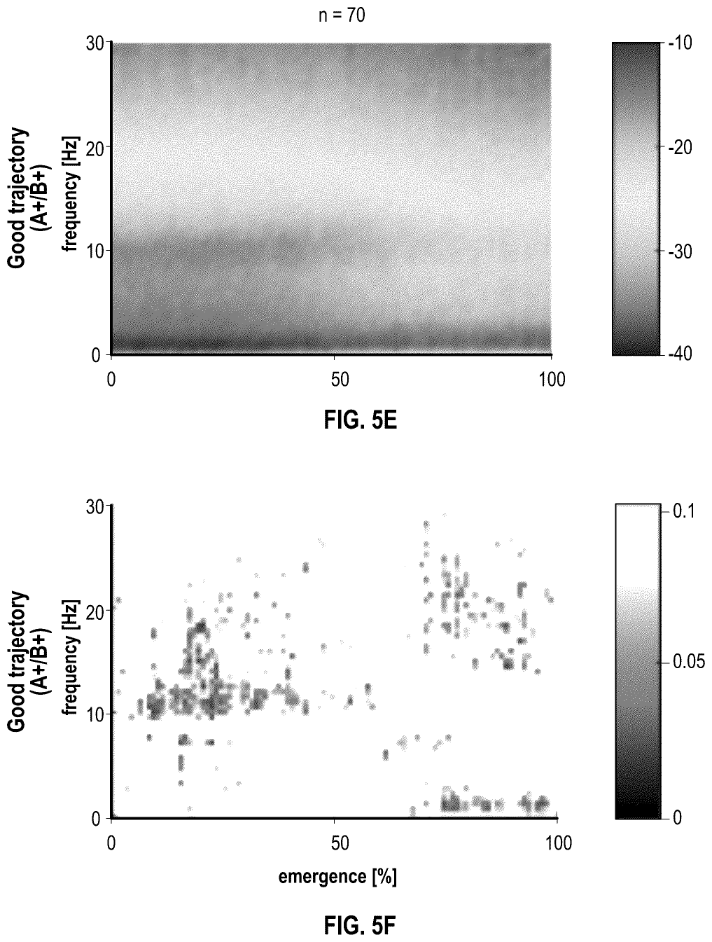

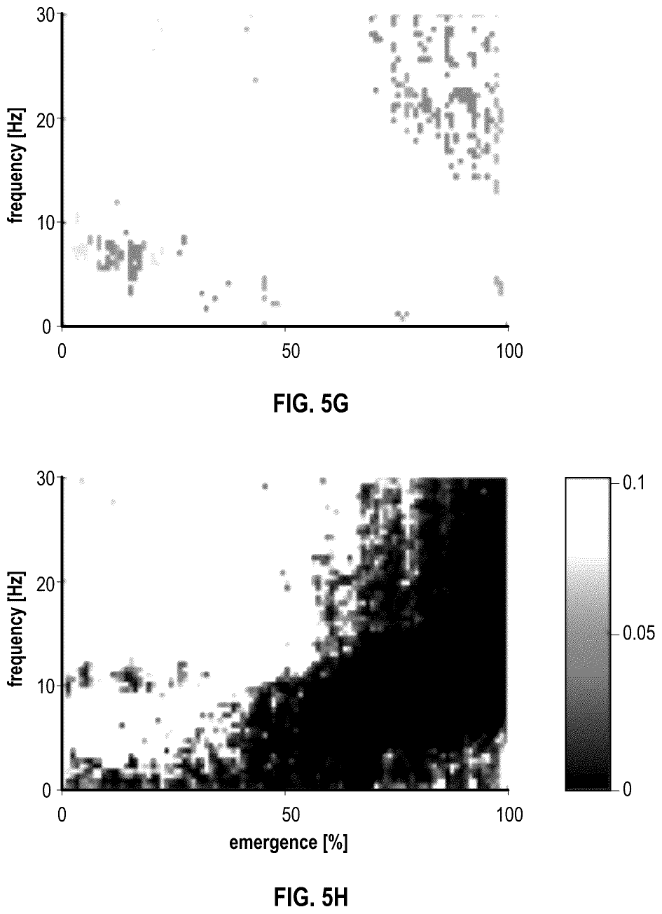

FIGS. 5A-5G shows exemplary cumulative normalized median spectrograms for the appropriate trajectory represented in the table illustrated in FIG. 19 by A−/B− and the bad trajectory (A+/B+) groups, each for patients with and without a positive CAM-ICU, according to the exemplary embodiments of the present disclosure. In particular, the exemplary cumulative normalized median spectrograms of FIGS. 5A-5C indicate the bad trajectory group, e.g., with Figure A showing positive CAM-ICU, Figue B showing negative CAM-ICU, and FIG. 5C showing spectral differences mostly in the alpha band during the first two thirds of emergence. FIGS. 5D-5E illustrate the good trajectory group of the exemplary cumulative normalized median spectrograms, with FIG. 5D showing positive CAM-ICU, and FIG. 5E illustrating negative CAM-ICU. FIG. 5F illustrates differences in the alpha band in the first half of emergence. In certain exemplary embodiments of the present disclosure, there can be significant difference across all bands between the good trajectory group and bad trajectory group in the second half of emergence for patients without a delirium (see, e.g., graph/illustration of FIG. 5H). There may be little or no significant differences in the group with a delirium between the good and bad trajectory group during emergence as shown in FIG. 5G.

### Exemplary Further Discussion

Exemplary embodiments of the present disclosure provide systems, methods and computer-accessible medium based on the slope of EEG power during emergence that can help to identify patients at low risk for a perioperative neurocognitive disorder, specifically delirium, and can be easily transferable into a clinical setting. (See, e.g., Ref. 7). The fact that patients can show a multitude of different EEG patterns during anesthesia emergence has been reported previously. (See, e.g., Refs. 17 and 18). The systems, methods and computer-accessible medium according to the exemplary embodiments of the present disclosure can classify different EEG emergence trajectories based on arbitrarily defined thresholds of EEG (band) power that allowed to classify the EEG into delta-dominant anesthesia, spindle dominant anesthesia, or non-slow-wave anesthesia. (See, e.g., Refs. 5 and 17). This exemplary approach can assist to relate the anesthesia emergence states to sleep states. (See, e.g., Ref. 17). However, a generalization of this concept to intraoperative monitoring can be complicated, as recovery from sleep and anethesia are different. In general, commercial EEG-based patient monitoring predominantly relies on quantitative changes in EEG band power or their ratios. (See, e.g., Refs. 19-21). Furthermore exemplary embodiments of the present disclosure can rely on the understanding that during emergence from general anesthesia induced by GABAergic agents patients transition from alpha oscillations to beta oscillations and the slow-wave delta oscillations should disappear in an uneventful case, which is termed a “zipper opening.” (See, e.g., Ref. 22). The consequence of this zipper opening behavior should lead to a universal decrease in power that exemplary embodiments describe as a favorable change.

Thus, the exemplary system, method and computer-accessible medium according to the exemplary embodiments of the present disclosure can evaluate the changes in total EEG power and absolute EEG band power during anesthesia emergence. During the transition from responsiveness to unresponsiveness/unconsciousness, the EEG changes from a fast and low-amplitude signal to slow and high-amplitude rhythmic activity. (See, e.g., Ref. 23). Although the loss and the return of responsiveness are not mirrored processes, the EEG should in the best case return to a the fast signal with low amplitude and a high frequency-amplitudes—as illustrated in exemplary embodiments of the present disclosure. (See, e.g., Ref. 24). If the EEG behaves differently, the patient can be at significantly higher risk to develop a delirium. Synchronized oscillations of neurons from UP to DOWN states are represented by high amplitude delta waves an are the most recognizable feature of general anesthesia. Higher frequency waves (Alpha and Beta) may reflect more sophisticated communication among cortical cells. Failure of cortical information processing of complex information may be part of the mechanism of delirium given that the differences between Delirium and no-Delirium groups can be mostly in the higher power oscillations.

The exemplary system, method and computer-accessible medium according to the exemplary embodiments of the present disclosure can indicate that the patient population with the lowest risk for a delirium exhibited a significant decrease in alpha band and beta band power throughout emergence, while patients exhibiting increasing power in either alpha or beta, or both, had a higher risk. In some exemplary embodiments, calculating risk differences between the higher risk groups may not be feasible, given a specific sample size. Exemplary embodiments of the present disclosure consider different combinations of bands. including alpha and delta. The exemplary system, method and computer-accessible medium according to the exemplary embodiments of the present disclosure can indicate that the combination of the alpha and the beta band yield can provide, e.g., the most promising results.

High intraoperative EEG alpha band power can be associated with an adequate anesthetic level (See, e.g., Refs. 25 and 26), and with the preoperative and perioperative cognitive state of the patient. (See, e.g., Refs. 27 and 28). In addition, during the anesthesia emergence, an episode of alpha-dominant activity seems beneficial for the patient. (See, e.g., Refs. 8 and 17). The systems, methods and computer-accessible medium according to the exemplary embodiments of the present disclosure that during a smooth emergence, the alpha power should fade and hence decrease as indicated by the negative slope. For the desired change in EEG beta band power, also a decrease, the explanation is not as straightforward. In fact, it seems counterintuitive, because strong beta band activity seems related to wakefulness. The systems, methods and computer-accessible medium according to the exemplary embodiments of the present disclosure can be used in a clinical setting in patients recovering from a surgical intervention. This inevitably leads to patients that at some point will start moving. EMG activity is known to influence clinical EEG recordings as its frequencies overlap with EEG. (See, e.g., Ref. 29). Further, the EMG can become more dominant in the higher frequencies, i.e., in the beta band/gamma band. The unfavorable increasing trend in beta band activity in exemplary embodiments may be caused by a more agitated patient. Patients with an endotracheal tube in place showed earlier signs of EMG activation than patients with a laryngeal mask. (See, e.g., Ref. 30). The systems, methods and computer-accessible medium according to the exemplary embodiments of the present disclosure can facilitate and account for EMG activity, as also integrated in the algorithms of the Entropy Module (GE. Helsinki, Finland). (See, e.g., Ref. 31).

There are other methods that try to classify anesthesia emergence in existence. For example, generic algorithm support vector machine approaches on EEG band power showed that emergence patterns are age specific. (See, e.g., Ref. 32). A linear curve fit is the easiest most economic approach and describes a general trend over a period of time, in this case the emergence phase. A sigmoidal/quadratic fit or a curve of higher order could provide a better fit during the interval of the emergence phase. However, these approaches could cause a higher number of resulting parameters as higher order curves are defined by a higher number of parameters. In this case an analysis with vector machines could be necessary, resulting in a need for a more sophisticated approach. Exemplary embodiments of the present disclosure utilize an approach which is easily transferable into a clinical setting, and thereby possibly offering the anesthesiologist an early sign for the neurocognitive outcome of the patient.

### Exemplary Limitations

Statistical trends presented by the exemplary system, method and computer-accessible medium according to the exemplary embodiments of the present disclosure may not be entirely based on the spectral information resulting from brain network function because the preoperative cognitive state of patients is not assessed apart from a delirium screening. First, it is known that patients with cortical atrophy are more likely to have lower total EEG power during maintenance and therefore also more likely to exhibit a flatter slope. Additionally, discontinuous EEG patterns indicative of excessive hypnotic administration (e.g., burst suppression) have low total EEG power and are not only more likely to occur in older patients, but they may also present a risk factor for a delirium, although in young and healthy subjects may not be affected. (See, e.g., Refs. 2, 3, and 33).

As discussed herein, a low total EEG power at end-maintenance can be associated with delirium and can contribute to flatter (or positive) slopes during emergence. Similarly, a longer time to emergence can contribute to a flatter slope during emergence and even though exemplary embodiments did not create a statistical association of time spent in emergence to a delirium, others have reported on this, and it may contribute to the strength of the exemplary embodiments' correlation of this parameter with delirium. (See, e.g., Ref. 17). These points are important to put into the context of the demonstrated correlation of this EEG parameter with delirium and therefore the frequency information should not be misinterpreted as a complete mechanistic explanation of the brain that is more susceptible to develop delirium. Furthermore, exemplary embodiments did not exclude patients with intraoperative burst suppression, which can also result in a lower power in the EEG. However, intraoperative burst suppression can also be associated with a higher risk for delirium, which points to the same results as shown in exemplary embodiments.

### Exemplary Conclusion

The systems, methods and computer-accessible medium according to the exemplary embodiments of the present disclosure can be provided to analyze a single frontal EEG channel and identify patterns highly specific for patients not at risk for a delirium. Using the systems, methods and computer-accessible medium according to the exemplary embodiments of the present disclosure, it is possible to economize resources concerning the screening of patients for a delirium in the PACU.

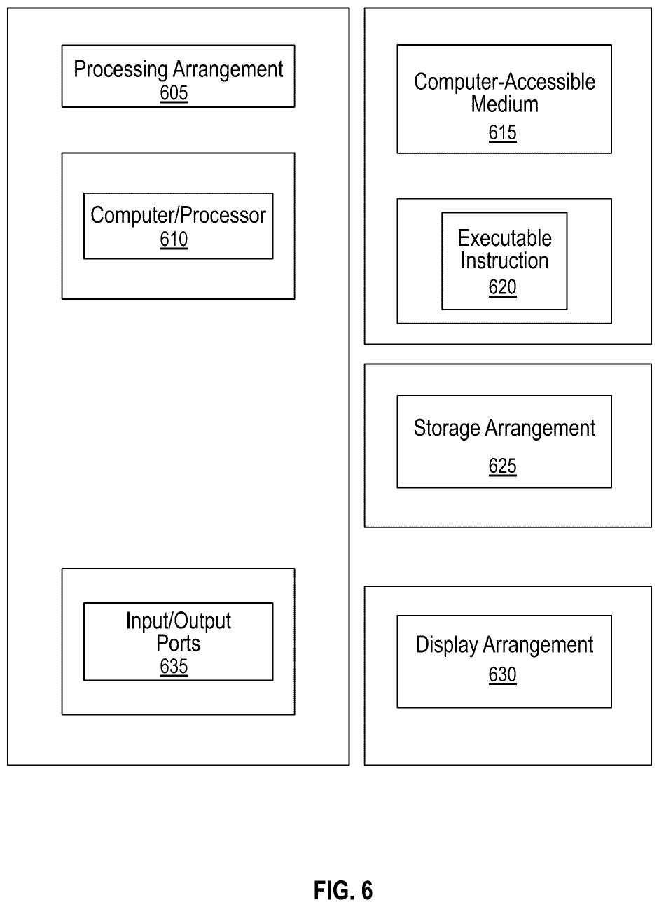

FIG. 6 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 605. Such processing/computing arrangement 605 can be, for example entirely or a part of, or include, but not limited to, a computer/processor 610 that can include, for example one or more microprocessors, and use instructions stored on a computer-accessible medium (e.g., RAM, ROM, hard drive, or other storage device).

As shown in FIG. 6, for example a computer-accessible medium 615 (e.g., as described herein above, a storage device such as a hard disk, floppy disk, memory stick, CD-ROM, RAM, ROM, etc., or a collection thereof) can be provided (e.g., in communication with the processing arrangement 605). The computer-accessible medium 615 can contain executable instructions 620 thereon. In addition or alternatively, a storage arrangement 625 can be provided separately from the computer-accessible medium 615, which can provide the instructions to the processing arrangement 605 so as to configure the processing arrangement to execute certain exemplary procedures, processes, and methods, as described herein above, for example. Further, the exemplary processing arrangement 605 can be provided with or include an input/output ports 635, which can include, for example a wired network, a wireless network, the internet, an intranet, a data collection probe, a sensor, etc. As shown in FIG. 6, the exemplary processing arrangement 605 can be in communication with an exemplary display arrangement 630, which, according to certain exemplary embodiments of the present disclosure, can be a touch-screen configured for inputting information to the processing arrangement in addition to outputting information from the processing arrangement, for example. Further, the exemplary display arrangement 630 and/or a storage arrangement 625 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### Exemplary Software: Age and Beta Entropy

The systems, methods and computer-accessible medium according to the exemplary embodiments of the present disclosure can be used for determining if a patient receiving analgo-sedative drugs has a brain that exhibits characteristics older or younger than their stated age. This can have obvious implications while maintaining general anesthesia during surgery where an anesthesiologist is titrating the concentration of hypnotic anesthetic agents to maintain unconsciousness during surgery. An overdose (or an underdose) may result in suboptimal care. Although for many decades anesthesiologists worried about an under-dose resulting in unintentional patient awareness, an overdose is now considered more common and just as problematic.

In addition to intraoperative hypotension, an overdose of many hypnotic medications have been associated with poor post-anesthesia outcomes such as delirium, delayed awakening, wound infection, mortality, increased length of stay, ICU admission, etc. It has been suggested that overdosing hypnotic medications may make the blood brain barrier more permeable allowing inflammatory cytokines access to the immune-privileged brain.

Severe overdoses of hypnotic medication can present as a discontinuous EEG (brief periods of low or absent synaptic activity at the cortex). Burst suppression is the most common discontinuous EEG pattern, but subtler titration of hypnotic agent may become especially necessary in patients that are older and at greater risk for subclinical cognitive impairment. Unfortunately, typical EEG patterns like burst suppression may not be visually detected in these patients—for this reason, exemplary embodiments of the present disclosure can consider the EEG by the entropy in the beta band and can suggest dose adjustments based on age, weight, and renal/hepatic function to optimize the delivery of analgo-sedative agents.

Like the operating room, any clinical scenario that involves patients receiving sedative drugs may benefit from the systems, methods and computer-accessible medium according to the exemplary embodiments of the present disclosure. This includes procedural sedation in the Emergency Department and in the Intensive Care Unit (ICU), where over 75% of patients are receiving sedative drugs for procedures or for improving comfort during mechanical ventilation. In the ICU, sedative agents are typically continued for days and in some cases weeks to months. It is critical to evaluate the patient's overall neurophysiologic activity in these situations and get the dosing correct to optimize care while avoiding unnecessary expense and/or complications. Like older patients presenting for surgery, ICU patients are often experiencing an acute clinical situation on top of a host of chronic conditions which may make estimations of dose difficult. Many patients suffer from systemic disease which can affect the clearance of analgo-sedative agents and the optimal brain dose can be affected by their acute state and medical condition. Therefore, utilizing the exemplary systems, method sand computer-accessible medium according to the exemplary embodiments of the present disclosure to determine whether a patient's brain responds to sedative medications more like an older or a younger patient can help an ICU team determine sedation strategy, prevent complications (e.g., delirium, hypotension), optimize care (e.g., shorter ICU stays) and aid in prognosis.

### Exemplary Special Use Case 1

In the epilepsy monitoring unit, patients are often not sedated while being continuously monitored with EEG. The exemplary system, method and computer-accessible medium according to the exemplary embodiments of the present disclosure can apply “age correction” based on beta entropy to the EEG signals recorded on epilepsy patients while they are sleeping in order to gain insight as to their overall cognitive health. Accordingly, the exemplary system, method and computer-accessible medium according to the exemplary embodiments of the present disclosure can provide and/or influence go-no go decisions on surgery and on the choice of anti-epileptic agents.

### Exemplary Software: Alpha Power Display

The systems, methods and computer-accessible medium according to the exemplary embodiments of the present disclosure can describe how different manipulations of the EEG can be helpful in determining the maximum alpha power while delivering analgo-sedative agents. Besides raw alpha power, oscillatory alpha and a visual examination of the first derivative of the EEG in the alpha band can indicate when a patient receives inadequate analgesic medication to suppress their brain's reaction to noxious stimulation. During surgery the surgical stimulation is not constant, at different times, the patient may need more or less pain medicine depending on the surgery. The loss of alpha power in an EEG may indicate a depolarization of the thalamus which precedes cortical activation due to sensory input detecting pain above a certain threshold. Unfortunately, because of changes in the EEG with age, it may not be possible to observe this phenomenon in frontal EEG recordings in older patients.

Due to cortical atrophy an increase in skull thickness, and age-related neurophysiologic changes, the EEG in older patients can have a more uniform spectral distribution of frequencies and may exhibit lower EEG amplitude. The systems, methods and computer-accessible medium according to the exemplary embodiments of the present disclosure can visually identify the response to noxious stimulation in these older patients by using the first derivative of the alpha power and the oscillatory component of alpha power. Therefore, examplary embodiments disclosed herein can be used by anesthesiologists interested in delivering adequate analgesic medication in patients of all ages undergoing surgery. In conjunction with age and beta entropy (discussed above), thresholds for switching between these different modalities can be identified.

The exemplary system, method and computer-accessible medium according to the exemplary embodiments of the present disclosure can indicate that strong alpha power during surgery has been associated with a decrease in delirium after surgery. Delirium is a major concern in the ICU because delirium is associated with poorer outcomes including death. Delirium (post-operative or otherwise) concerns family members and treating staff. It is also expensive by frequently leading to an escalation of care and longer lengths of stay. For example, strong alpha power may be present when the analgo-sedative regimen is giving adequate analgesia and non-excessive doses of hypnotic agents.

According to certain exemplary embodiments of the present disclsoure, the presence of alpha power can be associated with improved cognitive outcomes after surgery and it has been suggested that these improvements may also be seen in the ICU where a majority of patients are receiving both analgesic and sedative agents. Typically, ICU patients are older with severe co-morbidities, exemplary embodiments of the present disclosure can be used to guide sedation strategy and optimize dosing of sedative agents separately from analgesic agents, which can result in less delirium and a more efficient ICU stay.

### Exemplary Special Use Case 2

Patients with rarer disorders of sleep and arousal (e.g., TBI and post-coma patients) may benefit from applying the alpha visualization techniques of exemplary embodiments of the present disclosure during polysomnographic testing in order to subcategorize these disorders and may exemplary embodiments can aid in decisions on outpatient pharmacotherapy. Specifically, if a TBI patient reports inattentiveness during waking hours, according to exemplary embodiments, careful observation of the alpha power during sleep may make the difference between a neuropsychologist recommending methylphenidate (for attention deficit) vs modafinil (for inhibiting sleep inertia).

### Alternate Exemplary Electrode Configurations

Currently, most abbreviated (frontal) EEG monitors used during surgery or the ICU have a static configuration of electrodes (“patch”). Exemplary embodiments of the present disclosure describe how similar information could be gathered from alternate placing of electrodes which may be necessary, if for some reason the forehead cannot be used. This scenario is common in the operating room during facial, eye, ear, nose, or cranial surgery—but also potentially applicable in the ICU for example if a patient had facial burns or was in the prone position for lung protection.

### Exemplary Aperiodic Segment of EEG for Post-Operative Delirium Prediction

Not many prediction models exist for estimating the risk of post-operative delirium. Exemplary embodiments of the present disclosure provide a way to calculate the risk of the acute post-operative period to be complicated by delirium based on the intraoperative EEG signal, specifically by tracking the progression of the aperiodic component throughout the emergence period (from surgery end to regaining of consciousness). A clinician can use information provided by exemplary embodiments of the present disclosure to prepare for an escalation of care (e.g., request admission to intensive care level) and/or perhaps to avoid unnecessary imaging or tests that might be used to rule-out a reversible cause for a delay in return to normal cognition (e.g., emergency CT scan to rule-out intra-operative stroke).

### Exemplary Aperiodic Segment of EEG for Intraoperative Determination of Long-Term Cognitive Risk

Although some EEG biomarkers have been used as outpatient screening tools for diagnosing and/or risk stratification for developing neuro-cognitive decline (e.g., Alzheimer's and related dementias) in patients not receiving sedation—the interference of patient movement and EMG (electromyogram) artifact have prevented widespread clinical adoption. Exemplary embodiments of the present disclosure mitigate these problems and have the added benefit of evaluating the anesthetized brain which oscillates synchronously with large amplitude slow waves roughly indicative of cortical volume/size. By tracking how the broad spectrum (aperiodic) component of the EEG progresses through emergence, exemplary embodiments of the present disclosure can determine a very accurate metric of overall brain health. A clinician can use this information to inform the patient (or their family) about the overall risk of developing dementia and then might refer the patient for follow up with geriatric specialists.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example.” “in one exemplary embodiment.” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Throughout the disclosure, the following terms take at least the meanings explicitly associated herein, unless the context clearly dictates otherwise. The term “or” is intended to mean an inclusive “or.” Further, the terms “a.” “an,” and “the” are intended to mean one or more unless specified otherwise or clear from the context to be directed to a singular form.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### Exemplary References

The following references are hereby incorporated by reference, in their entireties:
1. Jin Z, Hu J, Ma D: Postoperative delirium: perioperative assessment, risk reduction, and management. Br J Anaesth 2020; 125; 492-504
2. Soehle M, Dittmann A, Ellerkmann R K, Baumgarten G, Putensen C, Guenther U: Intraoperative burst suppression is associated with postoperative delirium following cardiac surgery: a prospective, observational study. BMC Anesthesiology 2015; 15
3. Fritz B A, Kalarickal P L, Maybrier H R, Muench M R, Dearth D, Chen Y, Escallier K E, Ben Abdallah A, Lin N, Avidan M S: Intraoperative Electroencephalogram Suppression Predicts Postoperative Delirium. Anesthesia & Analgesia 2016; 122: 234-242
4. Georgii M T, Schneider G: [Deep anesthesia, poorly tolerated anesthesia?]. Anaesthesist 2019; 68: 583-593
5. Chander D, García P S, Maccoll J N, Illing S, Sleigh J W: Electroencephalographic Variation during End Maintenance and Emergence from Surgical Anesthesia. PLoS ONE 2014; 9: e106291
6. Hesse S, Kreuzer M, Hight D, Gaskell A, Devari P, Singh D, Taylor N B, Whalin M K, Lee S, Sleigh J W, Garcia P S: Association of electroencephalogram trajectories during emergence from anaesthesia with delirium in the postanaesthesia care unit: an early sign of postoperative complications. Br J Anaesth 2019; 122: 622-634
7. Evered L, Silbert B, Knopman D S, Scott D A, DeKosky S T, Rasmussen L S. Oh E S, Crosby G, Berger M, Eckenhoff R G, Nomenclature Consensus Working G: Recommendations for the Nomenclature of Cognitive Change Associated with Anaesthesia and Surgery-20181. J Alzheimers Dis 2018; 66: 1-10
8. Lutz R, Muller C, Dragovic S, Schneider F, Ribbe K, Anders M, Schmid S, Garcia PS, Schneider G, Kreuzer M, Kratzer S: The absence of dominant alpha-oscillatory EEG activity during emergence from delta-dominant anesthesia predicts neurocognitive impairment—results from a prospective observational trial. J Clin Anesth 2022; 82: 110949
9. Ely E W, Margolin R, Francis J, May L, Truman B, Dittus R, Speroff T, Gautam S, Bernard G R, Inouye SK: Evaluation of delirium in critically ill patients: validation of the Confusion Assessment Method for the Intensive Care Unit (CAM-ICU). Crit Care Med 2001; 29: 1370-9
10. Gusmao-Flores D, Salluh J I, Chalhub R A, Quarantini L C: The confusion assessment method for the intensive care unit (CAM-ICU) and intensive care delirium screening checklist (ICDSC) for the diagnosis of delirium: a systematic review and meta-analysis of clinical studies. Crit Care 2012; 16: R115
11. Chernik D A, Gillings D, Laine H, Hendler J, Silver J M, Davidson A B, Schwam E M, Siegel J L: Validity and reliability of the Observer's Assessment of Alertness/Sedation Scale: study with intravenous midazolam. J Clin Psychopharmacol 1990; 10:244-51
12. Delorme A, Makeig S: EEGLAB: an open source toolbox for analysis of single-trial EEG dynamics including independent component analysis. Journal of Neuroscience Methods 2004; 134: 9-21
13. Bonhomme V, Hans P: Muscle relaxation and depth of anaesthesia: where is the missing link? Br J Anaesth 2007; 99: 456-60
14. Goncharova, II, McFarland D J, Vaughan T M, Wolpaw J R: EMG contamination of EEG: spectral and topographical characteristics. Clin Neurophysiol 2003; 114: 1580-93
15. Chang C Y, Hsu S H, Pion-Tonachini L, Jung T P: Evaluation of Artifact Subspace Reconstruction for Automatic Artifact Components Removal in Multi-Channel EEG Recordings. IEEE Trans Biomed Eng 2020; 67: 1114-1121
16. Hentschke H, Stüttgen MC: Computation of measures of effect size for neuroscience data sets. European Journal of Neuroscience 2011; 34: 1887-1894
17. Hesse S, Kreuzer M, Hight D, Gaskell A, Devari P, Singh D, Taylor N, Whalin M, Lee S, Sleigh J: Association of electroencephalogram trajectories during emergence from anaesthesia with delirium in the post-anaesthesia care unit: an early sign of postoperative complications. British Journal of Anaesthesia 2019; 122: 622-634
18. Chander D, Garcia P S, MacColl J N, Illing S, Sleigh J W: Electroencephalographic variation during end maintenance and emergence from surgical anesthesia. PLOS One 2014; 9: e106291
19. Rampil I J: A Primer for EEG Signal Processing in Anesthesia. Anesthesiology 1998; 89: 980-1002
20. Jensen E W, Valencia J F, Lopez A, Anglada T, Agusti M, Ramos Y, Serra R, Jospin M, Pineda P, Gambus P: Monitoring hypnotic effect and nociception with two EEG-derived indices. qCON and qNOX, during general anaesthesia. Acta Anaesthesiol Scand 2014; 58: 933-41
21. Drover D. Ortega HR: Patient state index. Best Practice & Research Clinical Anaesthesiology 2006; 20: 121-128
22. Purdon P L, Sampson A, Pavone K J, Brown E N: Clinical Electroencephalography for Anesthesiologists: Part I: Background and Basic Signatures. Anesthesiology 2015; 123: 937-60
23. Purdon P L, Pierce E T, Mukamel E A, Prerau M J, Walsh J L, Wong K F K, Salazar-Gomez A F, Harrell P G, Sampson A L, Cimenser A: Electroencephalogram signatures of loss and recovery of consciousness from propofol. Proceedings of the National Academy of Sciences 2013; 110: E1142-E1151
24. Hudetz A G: General anesthesia and human brain connectivity. Brain connectivity 2012; 2: 291-302
25. Hight D F, Gaskell A L, Kreuzer M, Voss L J, Garcia P S, Sleigh J W: Transient electroencephalographic alpha power loss during maintenance of general anaesthesia. British journal of anaesthesia 2019; 122: 635-642
26. Hagihira S, Takashina M, Mori T, Ueyama H, Mashimo T: Electroencephalographic bicoherence is sensitive to noxious stimuli during isoflurane or sevoflurane anesthesia. The Journal of the American Society of Anesthesiologists 2004; 100: 818-825
27. Giattino C, Gardner J, Sbahi F, Roberts K, Cooter M, Moretti E, Browndyke J, Mathew J, Woldorff M, Berger M: Intraoperative Frontal Alpha-Band Power Correlates with Preoperative Neurocognitive Function in Older Adults. Frontiers in Systems Neuroscience 2017; 11
28. Gutierrez R, Egana J I, Saez I, Reyes F, Briceno C, Venegas M, Lavado I, Penna A: Intraoperative Low Alpha Power in the Electroencephalogram Is Associated With Postoperative Subsyndromal Delirium. Front Syst Neurosci 2019; 13: 56
29. Kamata K, Aho A, Hagihira S, Yli-Hankala A, Jäntti V: Frequency band of EMG in anaesthesia monitoring. British journal of anaesthesia 2011; 107: 822-823
30. Hight D F, Voss L J, Garcia P S, Sleigh J W: Electromyographic activation reveals cortical and sub-cortical dissociation during emergence from general anesthesia. J Clin Monit Comput 2017; 31: 813-823
31. Viertio-Oja H, Maja V, Sarkela M, Talja P, Tenkanen N, Tolvanen-Laakso H, Paloheimo M, Vakkuri A, Yli-Hankala A, Merilainen P: Description of the Entropy algorithm as applied in the Datex-Ohmeda S/5 Entropy Module. Acta Anaesthesiol Scand 2004; 48: 154-61
32. Liang Z, Huang C, Li Y, Hight D F, Voss L J, Sleigh J W, Li X, Bai Y: Emergence EEG pattern classification in sevoflurane anesthesia. Physiol Meas 2018; 39: 045006
33. Shortal B P, Hickman L B, Mak-McCully R A, Wang W, Brennan C, Ung H, Litt B, Tarnal V, Janke E, Picton P, Blain-Moraes S, Maybrier H R, Muench M R, Lin N, Avidan M S, Mashour G A, Mckinstry-Wu A R, Kelz M B, Palanca B J, Proekt A, Re CSG: Duration of EEG suppression does not predict recovery time or degree of cognitive impairment after general anaesthesia in human volunteers. Br J Anaesth 2019; 123: 206-218
34. Banerji A, Sleigh J W, Voss L J, Garcia P S, Gaskell A L: Deconstructing delirium in the post anaesthesia care unit. Front Aging Neurosci 2022; 14: 930434

What is claimed is:

1. A method for generating diagnostic data associated with a likelihood of developing neurocognitive impairment, comprising:
monitoring electroencephalography (EEG) data from the patient during an emergence from a general anesthesia previously provided to the patient; and
predicting neurocognitive impairment based on a slope of EEG power.

2. The method of claim 1, wherein the neurocognitive impairment is delirium.

3. The method of claim 1, wherein the neurocognitive impairment is a long-term impairment.

4. The method of claim 3, wherein the long-term impairment is Alzheimer's.

5. The method of claim 1, further comprising:
directing a medical intervention based on the generated diagnostic data.

6. The method of claim 5, wherein the medical intervention is an order for continued monitoring for neurocognitive impairment.

7. The method of claim 5, wherein the medical intervention is an order for a brain scan.

8. A system for generating diagnostic data associated with a likelihood of developing neurocognitive impairment, comprising:
a processor configured to:
monitor electroencephalography (EEG) data from the patient during an emergence from a general anesthesia previously provided to the patient, and
predict neurocognitive impairment based on a slope of EEG power.

9. The system of claim 8, wherein the neurocognitive impairment is delirium.

10. The system of claim 8, wherein the neurocognitive impairment is a long-term impairment.

11. The system of claim 10, wherein the long-term impairment is Alzheimer's.

12. The system of claim 8, further comprising:
directing a medical intervention based on the generated diagnostic data.

13. The system of claim 12, wherein the medical intervention is an order for continued monitoring for neurocognitive impairment.

14. The system of claim 12, wherein the medical intervention is an order for a brain scan.

15. A computer-readable non-transitory medium comprising computer-executable instructions for generating diagnostic data associated with a likelihood of developing neurocognitive impairment, wherein, when executed by at least one computer processor, the computer-executable instructions configure the at least one pcomputer processor to perform procedures comprising:
monitoring electroencephalography (EEG) data from the patient during an emergence from a general anesthesia previously provided to the patient; and
predicting neurocognitive impairment based on a slope of EEG power.

16. The computer-readable non-transitory medium of claim 15, wherein the neurocognitive impairment is delirium.

17. The computer-readable non-transitory medium of claim 15, wherein the neurocognitive impairment is a long-term impairment.

18. The computer-readable non-transitory medium of claim 17, wherein the long-term impairment is Alzheimer's.

19. The computer-readable non-transitory medium of claim 15, wherein the at least one computer processor is further configured to perform at least one procedure comprising:
directing a medical intervention based on the generated diagnostic data.

20. The computer-readable non-transitory medium of claim 19, wherein the medical intervention is an order for continued monitoring for neurocognitive impairment.

21. The computer-readable non-transitory medium of claim 19, wherein the medical intervention is an order for a brain scan.
